(19) United States
(12) Patent Application Publication
(10) Pub. No.: US 2007/0287988 A1
(43) Pub. Date: Dec. 13, 2007

(54) Method and composition for treating osteonecrosis and/or avascular necrosis

(76) Inventors: Linda M. Trebing, Madison, NJ (US); James P. Seaton, Chatham, NJ (US)

Correspondence Address: Melvin I. Stoltz, 51 Cherry Street, Milford, CT, 06460, US

(21) Appl. No.: 11/729,231

(22) Filed: Mar. 28, 2007

Related U.S. Application Data
(60) Provisional application No. 60/791,542, filed on Apr. 12, 2006.

(51) Int. Cl. A61F 2/82; A61K 31/765; A61P 19/08

(52) U.S. Cl. 604/506; 424/78.37

(57) ABSTRACT

By providing an elastic form stable material which is capable of being delivered directly to a specific desired location within a living creature and provides increased strength and rigidity to the injected location, disorders of the trabecular bone or subchondral tissue of a living creature are able to be effectively treated. Treatment of necrosis of the bone beneath the cartilage in skeletal joints and other defects in bony or cartilaginous structures is achieved with a variation of the subject material specific to each application. In the preferred method, the elastic form stable material is injected directly into the affected area, thereby achieving the desired result.

### RELATED APPLICATIONS

This application is related to U.S. Provisional Patent Application Ser. No. 60/791,542, filed Apr. 12, 2006, entitled METHOD AND COMPOSITION FOR TREATING OSTEONECROSIS AND/OR AVASCULAR NECROSIS.

### TECHNICAL FIELD

The present invention relates to a method of injecting a polymer to treat disordered, insufficient, or injured structures in a living creature, in particular, a human being. This invention also relates to a composition suitable for use in such a method, its preparation and use.

### BACKGROUND ART

Many structures in the human body rely upon the flexibility or elasticity of the tissue to exhibit the preferred properties or to perform a desired task. The resilience of the tissue often provides a means for better load distribution and shock-absorbing characteristics. The elasticity may function to create a more natural appearance or more desirable tactile property. Specific examples of these types of structures include the intervertebral discs, the substrate underlying cartilaginous surfaces, or the connective and soft tissue beneath the skin. The properties of these tissues are associated with providing shape and resilience for functionality of joints and load-bearing surfaces or for giving form to external, visible parts of the body.

Osteonecrosis or avascular necrosis is characterized by areas of dead trabecular bone, often involving the subchondral plate. This degradation of bone underlying articular and load-bearing surfaces in the body can cause loss of support for the cartilage, leading to severe deterioration of the joint.

Severe joint destruction resulting from osteonecrosis is seen in 50% of the patients, and a major surgical procedure is often required within 3 years of diagnosis. It is estimated that almost 10% of the 500,000 total hip replacements performed annually in the United States are intended to treat AVN.

### Osteonecrosis

Osteonecrosis, or AVN (Avascular Necrosis), is a condition in which the underlying bone beneath an articular surface begins to atrophy and collapse, causing pain and instability. It most commonly affects the hip joint and can often occur in younger patients before the cartilage is worn enough to require a total or partial joint replacement. After the supporting bone under an articular surface has degraded, damage to the cartilage often follows, resulting in a painful, unstable joint. Current treatment is primarily limited to debridement and autologeous bone grafting, which is technically challenging in which the surgeon harvests cancellous bone from the patient, requiring a second incision and co-morbidity. Replacement of the femoral head or total joint arthroplasty is needed if the bone grafting procedure is unsuccessful or contraindicated.

In spite of the need that has long existed for improved treatment in this area, no substantive advancements have been made. As a result, a long felt need exists for both compositions and methods of use which would allow the repair of damaged bone areas without the need for extensive surgery or joint replacement.

### SUMMARY OF THE INVENTION

By employing the present invention, all of the difficulties and drawbacks found in the prior art have been eliminated and a highly effective method for treating avascular necrosis or osteonecrosis in living creatures is attained. In addition, the present invention also achieves a unique composition particularly formulated and suitable for use in the method of the present invention.

In accordance with the teaching of the present invention, a physician or clinician injects a flowable substance into the necrotic bone which polymerizes in situ, filling the voids and defects in the bone and providing support to the cartilage and surrounding bone. It has been found that by injecting the flowable substance into the bone below the surface, small voids are filled and support is provided for the articulating surface, such as the femoral head. Furthermore, the polymerized material adds strength to the structure supporting the cartilage. By properly selecting the flowable, polymerizable material with a particular balance of elastic and compressive properties, along with early intervention, a minimally invasive procedure is employed which provides pain relief and increased mobility.

By employing the present invention, immediate stabilization and strengthening of the affected area is achieved. In the prior art method of bone grafting and hip replacement painful recovery periods with limited weight bearing ability are typical. The proposed material and method allows the patient to return to normal daily activities earlier and delays the need for total hip arthroplasty for a length of time without affecting the outcomes of future therapies.

The material employed in the present invention polymerizes at approximate body temperature, therefore reducing or eliminating the risk of further necrosis of surrounding bone due to an exothermic reaction, as is often exhibited in the use of polymethyl methacrylate bone cement. Additionally, active agents, such as bone morphogenic proteins, can be added to the injected material to promote bone growth or treat the area urrounding the osteonecrotic bone.

The method of the present invention comprises the injection of an elastomeric filler into the structure to be treated via a percutaneous route, usually under X-Ray guidance, such as lateral projection fluoroscopy. The material is injected as a paste or semi-liquid from a suitable gun or injection system via a needle that has been passed into the body to apply the material to the affected area.

The elastomeric filler, once injected, will polymerize in situ. The resulting material provides reinforcement to or replacement of tissue or anatomical structures that are deficient due to the aging process, tumor removal or other surgical intervention or trauma. In addition to the reinforcing, strengthening and shock-absorbing properties it is desirable that the starting filler composition is of a viscosity that allows it to flow into the voids or spaces as required.

Therefore, it is a principal object of the present invention to provide filler material and a method for delivering the filler material into the substrate beneath the cartilage in articulating joints and filling the voids in bony or cartilaginous structures created or treated during plastic or neurosurgical procedures, or other similar applications, which is easily prepared and delivered to the affected area while also providing the desired filling, reinforcing, strengthening and shock-absorbing properties.

Another object of the present invention is to provide filler material and a method for using the filler material in the affected areas having the characteristic features described above which is inherently flexible and viscous to provide flowability throughout the structures as required both during its application and after curing, thereby achieving self-regulating control realized from the fluid properties of the injected liquid and the elastomeric characteristics of the polymerized material.

Another object of the present invention is to provide filler material and a method for using the filler material in the body having the characteristic features described above which is capable of being prepared to exhibit varying levels of hardness or stiffness after curing, thereby allowing the selection and formulation of the filler material with appropriate mechanical properties specifically suited for each application.

Another object of the present invention is to provide filler material and a method for using the filler material in the body having the characteristic features described above which is capable of being prepared with additives that remain active for a length of time after curing, thereby allowing localized therapeutic treatment of the affected area or anatomic structure.

Another object of the present invention is to provide filler material and a method for using the filler material having the characteristic features described above which enables the application of the filler material in the body in a controlled and directed manner that results in the placement of the material in targeted areas and having a specific geometry that is preferred for the treatment modality.

Another object of the present invention is to provide filler material and a method for using the filler material having the characteristic features described above which enables the application of the material into the femoral head in a manner which allows surgeons to utilize familiar targeting devices and drill guides to place the needle or cannula to inject the flowable material, since such drill guide systems are commonly used by orthopedic and trauma surgeons in the implantation of hip screws and hip screw and plate systems for the treatment of femoral neck fractures.

Other and more specific objects will in part be obvious and will in part appear hereinafter.

### THE DRAWINGS

For a fuller understanding of the nature and objects of the invention, reference should be had to the following detailed description taken in connection with the accompanying drawings, in which:

FIG. 1 is a perspective view of a normal femur bone with a healthy femoral head;

FIGS. 2-4 are perspective views, partially broken away, depicting a femur bone with a femoral head having varying stages of bone necrosis; and

FIGS. 5-8 are perspective views, partially broken away, depicting a femur bone with a femoral head having bone necrosis in progressive stages of being repaired using the process of the present invention.

### DETAILED DISCLOSURE

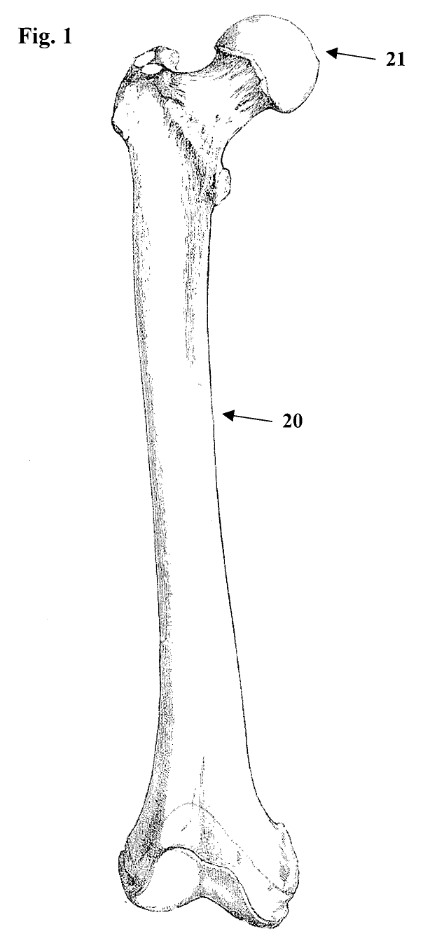

By referring to FIGS. 1-8, along with the following detailed disclosure, one principal treatment method of the present invention can best be understood. In this regard, as detailed above, the present invention can be implemented in many areas of a human body. However, one principal application of the present invention is in the repair of bone necrosis in the femoral head of a femur. In addition, the unique formulation of the material employed in the method of the present invention is also fully disclosed. However, alterations or variations in both the method and the formulation of the preferred material can be made without departing from the scope of the present invention. Consequently, it is to be understood that the following detailed discussion and the drawings are provided for exemplary purposes only and are not intended as a limitation of the present invention.

In FIG. 1, a normal femur bone 20 is depicted, which incorporates healthy femoral head 21. As is well known, femoral head 21 of femoral bone 20 cooperates with the hip bone of an individual to form a ball and socket construction which enables the leg of an individual to articulate in the desired manner. Although not shown, femoral head 21 of femoral bone 20 is surrounded by cartilage and held in place by muscle tissue.

Due to various conditions which may exist with an individual, the bone tissue forming femoral head 21 may die, causing the bone to collapse. Depending upon the part of the bone that is affected and how large an area is involved, substantial disability may result: In addition, pain is often present which usually develops and increases in severity.

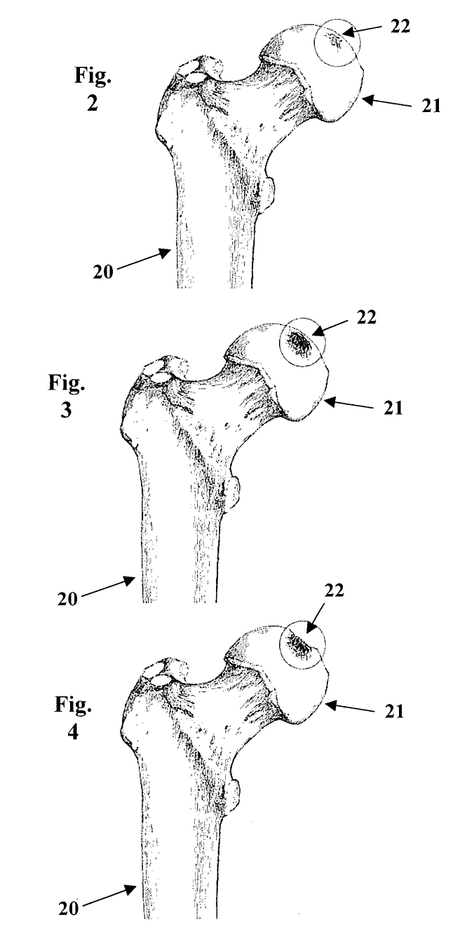

In FIGS. 2-4, various stages of bone necrosis of femoral head 21 are depicted. In FIG. 2, area or site 22 shows the early stage of bone necrosis, while FIG. 3 depicts advanced bone necrosis in area/site 22. Finally, FIG. 4 shows a worsened condition in area/site 22 depicting the loss of the underlying bone of the femoral head due to osteonecrosis.

As the damage caused by avascular necrosis continues to advance, most patients experience joint pain which develops and may be mild or severe. However, if avascular necrosis progresses in the bone and the surrounding joint surface collapses, particularly as shown in FIG. 4, pain may develop and increase dramatically, with the pain possibly being severe enough to limit the individual's range of motion in the affected joint.

By employing the present invention, any damage caused by bone necrosis is capable of being repaired in a direct, easily implemented process, providing an individual with increased mobility, pain reduction, and additional time without requiring invasive surgical procedures. As detailed herein, a cannula is inserted into the bone with the distal end thereof positioned in the area of the damage. Thereafter, a uniquely formulated, curable, flowable, filler composition is injected through the cannula to the damaged bone area. Once the flowable material has filled the bone voids created by the avascular necrosis, the material is allowed to polymerized in situ. Once fully polymerized, the material provides strength, rigidity, and resilient support to the surface area which had previously been damaged. In this way, all of the adverse effects created by bone necrosis can be substantially eliminated with a procedure which avoids typical invasive surgery.

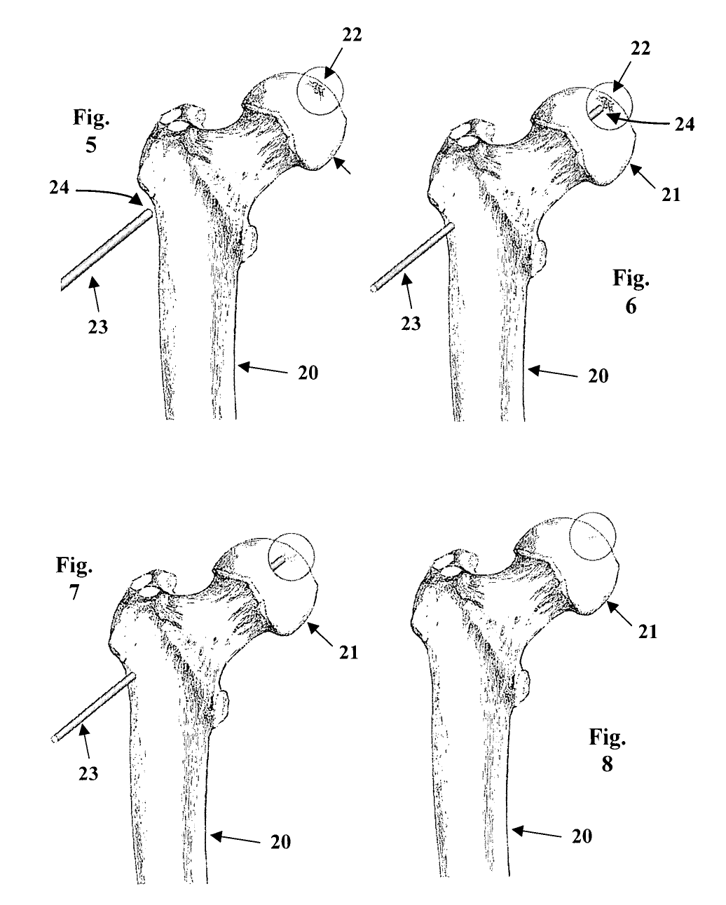

By referring to FIGS. 5-8, along with the following detailed discussion, the method of the present invention can best be understood. In this regard, the following discussion focuses upon the use of the present invention in repairing the femoral head of a femur bone, since this is a typical area adversely affected by bone necrosis. However, since numerous other areas in the human body exist which are damaged by bone necrosis, it is to be understood that this disclosure is provided for exemplary purposes only and is not intended as a limitation of the present invention.

In accordance with the present invention, the curable, flowable, filler composition is delivered to area or site 22 affected by bone necrosis through cannula 23. Depending upon the particular site or area 22 to be treated, a cavity or passageway is drilled in femur bone 20 to enable cannula 23 to be inserted into the bone in cooperating association with area/site 22. In the embodiment depicted in FIGS. 5-8, the cavity or passageway is drilled in femur 20 from the opposed end of area 22, in order to enable cannula 23 to be inserted through the passageway and allow the distal end 24 of cannula 23 to be brought into juxtaposed, spaced, cooperating association directly below area/site 22 and the surface of femoral head 21.

As shown, once distal end 24 of cannula 23 is placed directly below the damaged area/site 22, the desired curable, flowable, filler composition, which comprises an elastic form stable material, is injected into cannula 23 in order to allow the filler material to exit from distal end 24 of cannula 23 and flow into damaged area/site 22. As the curable, flowable, filler material is delivered to damaged area/site 22, the filler material effectively fills the voids created by the bone deterioration, restoring the area and providing support for the articulating surface thereof.

As shown in FIG. 7, cannula 23 remains in femur 20 as the filler material is allowed to polymerized in situ, filling in the bone defects originally present in femoral head 21. Once the filler material has been substantially fully polymerized and it has been determined that additional filler material is not needed, cannula 23 is removed, as depicted in FIG. 8, leaving the fully polymerized filler material effectively reconstructing the damaged area of femoral head 21, providing resilient support for the cartilage positioned between femur 20 and its associated hip bone.

In accordance with the present invention, it has been found that the preferred curable filler composition comprises an elastic form stable material. Preferably, this material comprises a silicone elastomer, with poly (dimethyl siloxane) being preferred. In addition, in the preferred formulation, the composition also incorporates a cross-linking agent and a diluent.

One composition of the curable filler material of the present invention which has been found to be extremely successful comprises between about 60% and 85% by weight based upon the weight of the entire composition of poly (dimethyl siloxane), between about 2% and 5% by weight based upon the weight of the entire composition of a cross-linking agent, and between about 10% and 20% by weight based on the weight of the entire composition of a diluent. In addition, if desired, a radiopaque material may be incorporated into the composition in order to enable the delivery of the material into the bony tissue to be monitored by using suitable equipment, such as x-rays.

In this regard, it has been found that between about 100% and 20% by, weight based upon the weight of the entire composition of the radiopaque material is preferably employed. In addition, the radiopaque material preferably comprises one selected from the group consisting of silver powder, barium sulfate, bismuth trioxide, zirconium dioxide, tantalum or titanium powders or fibers, calcium sulfate, calcium phosphate, hydroxyapetite, tricalcium phosphate, and other medically appropriate opacifiers or agents. It has also been found that the curable filler material employed in the method of the present invention preferably possesses a durometer in the cured state which ranges between about 10 A and 90 A.

One preferred formulation of the “cure-in-place” silicone elastomer of the present invention comprises two highly viscous liquid components, namely reinforced dimethyl methylvinyl siloxanes and reinforced dimethyl methylhydrogen siloxanes, supplied in equal parts (1:1 ratio). In addition, this preferred composition is preferably supplied in a pre-filled, two-part mixing and dispensing cartridge/syringe system wherein the two viscous liquid components are maintained separately until the time of use. When desired, the components are thoroughly intermixed with each other in the desired uniform ratio to achieve the desired uniform consistency.

Once the mixing process has been completed, the resulting silicone elastomer is immediately injected into the desired site wherein the material polymerizes in situ in approximately 3 to 15 minutes. Once cured, the silicone elastomer results in a tough, rubbery consistency which has low toxicity and presents a low risk of unfavorable biological reactions. In addition, the preferred formulation preferably incorporates a radio-opaque material in order to enable the delivery of the material to be monitored with standard fluoroscopy.

In accordance with the present invention, it has been found at the following compositions represents the preferred formulations for the two component system of this invention:

Component A:

Vinyldimethyl terminated dimethyl polysiloxane/trimethylsiloxy terminated polydimethyl siloxane, 64%

Silica, amorphous, 21%

Barium Sulfate powder, USP, 105%

Pt Catalyst >0.001%

Component B:

Vinyldimethyl terminated dimethyl polysiloxane/trimethylsiloxy terminated polydimethyl siloxane, 630%

Silica, amorphous, 21%

Barium Sulfate powder, USP, 15%

Trimethyl methyl-hydro dimethyl siloxane (crosslinker), 1%

### By employing the compositions detailed above, all of the desired goals and objectives of the present invention are realized.

In addition, it has also been found that the preferred curable filler composition may incorporate bioactive compounds in order to provide further enhanced medicinal beneficial effects. In this regard, the preferred bioactive compound incorporated into the filler composition comprises one or more selected from the group consisting of antibiotics, antimicrobial agents, tumor therapy compounds, radioactive isomers, chemotherapy substances, local anesthetic compounds, steroids, and other medically appropriate bioactive agents.

In the process of delivering the curable filler material detailed above to the desired site, it has been found that the filler material may be injected in two or more stages. In this regard, in the first stage, the initial defects or voids in the trabecular bone of the femoral head are filled in order to reinforce or strengthen a weakened area of the femoral head. In this regard, the durometer of the resulting cured material is preferably selected to provide increased strength and stability to the trabecular bone in the femoral head.

In the second stage of the process of the present invention, areas of the femoral head directly beneath the cartilage are filled, in order to provide enhanced support to the articular surface. It has been found that the second stage injection may be introduced after the first stage of material has cured or, if desired, may be performed during a subsequent procedure.

In addition, it has also been found that the durometer of the resulting cured material injected in the second stage is preferably selected to provide increased flexibility, resistance, or a greater capacity to distribute the forces seen by the articulating surface and providing its shock observing characteristics. Although not required, it is often found that the durometer of the cured material injected in the second stage is different from the durometer of the material injected in the first stage.

It will thus be seen that the object set forth above, among those made apparent from the preceding description, are efficiently attained and, since certain changes may be made in carrying out the above method and in the composition set forth without departing from the scope of the invention, it is intended that all matter contained in the above description or shown in the accompanying drawing shall be interpreted as illustrative and not in a limiting sense.

It is also to be understood that the following claims are intended to cover all of the generic and specific features of the invention herein described, and all statements of the scope of the invention which, as a matter of language, might be said to fall there between.

Particularly, it is to be understood that in said claims, ingredients or compounds recited in the singular are intended to include compatible mixtures of such ingredients wherever the sense permits.

Having described our invention, what we claim as new and desire to secure by Letters Patent is:

1. An injection method for treating diseased, deficient or injured bone in a living creature, in particular a human being exhibiting osteonecrosis or avascular necrosis, comprising the step of injecting a curable filler composition in said bony tissue, wherein the curable filler material comprises an elastic form stable material.

2. The method defined in claim 1, wherein said composition comprises a curable elastomer-precursor composition.

3. The method defined in claim 2, wherein said composition comprises a silicone elastomer.

4. The method defined in claim 3, wherein said silicone elastomer comprises poly(dimethyl siloxane).

5. The method defined in claim 4, wherein said composition additionally comprises a cross-linking agent and a diluent.

6. The method defined in claim 5, wherein said composition comprises a radiopaque material.

7. The method defined in claim 6, wherein said radiopaque material comprises one selected from the group consisting of silver powder, barium sulfate, bismuth trioxide, zirconium dioxide, tantalum or titanium powders or fibers, calcium sulfate, calcium phosphate, hydroxyapetite, tri calcium phosphate, and other medically appropriate opacifier agents.

8. The method defined in claim 7, wherein curable filler material is further defined as comprising:
A. between about 60% and 85% by weight based upon the weight of the entire composition of poly(dimethyl siloxane);
B. between about 2% and 5% by weight based upon the weight of the entire composition of the cross-linking agent;
C. between about 100% and 20% by weight based upon the weight of the entire composition of the diluent; and
D. between about 100% and 20% by weight based upon the weight of the entire composition of the radiopaque material.

9. The method defined in claim 8, wherein said composition is prepared in advance in a mixing-dispensing device.

10. The method defined in claim 8, wherein said composition is delivered to the femoral head by inserting a needle into an internal cavity formed in the femoral head and causing the filler material to flow through the needle into the femoral head.

11. The method defined in claim 10, comprising the additional steps of stopping the flow of the filler material when the affected area of the femoral head has been filled with the filler material, and thereafter withdrawing the needle from the femoral head.

12. The method defined in claim 8, wherein the curable filler is formulated to possess a durometer in the cured state which ranges between about 10 A and 90 A.

13. The method defined in claim 8, wherein said filler material is further defined as being flexible, when cured, to move, shift, compress, and or elongate within the structure of the femoral head and the voids, cracks or weakened areas, thereby providing varying actions or reactions.

14. The method defined in claim 5, wherein said composition comprises bioactive compounds selected from the group consisting of antibiotics, anti-microbial agents, tumor therapy compounds, radioactive isomers, chemotherapy substances, local anesthetic compounds steroid, and other medically appropriate bioactive agents.

15. The method defined in claim 14, wherein said composition comprises a radiopaque material.

16. The method defined in claim 15, wherein said radiopaque material comprises one selected from the group consisting of silver powder, barium sulfate, bismuth trioxide, zirconium dioxide, tantalum or titanium powders or fibers, calcium sulfate, calcium phosphate, hydroxyapetite, tri calcium phosphate, and other medically appropriate opacifier agents.

17. The method defined in claim 16, wherein curable filler material is further defined as comprising:
A. between about 60% and 85% by weight based upon the weight of the entire composition of poly(dimethyl siloxane);
B. between about 2% and 5% by weight based upon the weight of the entire composition of the cross-linking agent;
C. between about 100% and 20% by weight based upon the weight of the entire composition of the diluent; and
D. between about 100% and 20% by weight based upon the weight of the entire composition of the radiopaque material.

18. The method defined in claim 17, wherein said composition is prepared in advance in a mixing-dispensing device.

19. The method defined in claim 16, wherein said composition is delivered to the femoral head by inserting a needle into the internal cavity of the femoral head and causing the filler material to flow through the needle into the femoral head.

20. The method defined in claim 19, comprising the additional steps of stopping the flow of the filler material when the affected area of the femoral head has been filled with the filler material, and thereafter withdrawing the needle from the femoral head.

21. The method defined in claim 17, wherein the curable filler is formulated to possess a durometer in the cured state which ranges between about 10 A and 90 A.

22. The method defined in claim 17, wherein said filler material is further defined as being flexible, when cured, to move, shift, compress, and or elongate within the structure of the femoral head and the voids, cracks or weakened areas, thereby providing varying actions or reactions.

23. A method of preparing a composition for injection into a disordered or diseased area of the body of a living creature, in particular a human being, said composition comprising an elastic form stable material consisting of a curable elastomer-precursor composition and additives intermixed therewith and possesses substantially reduced toxicity, thereby enabling its use prophylactically.

24. The method defined in claim 23, wherein the curable elastomer-precursor composition comprises a silicone elastomer.

25. The method defined in claim 24, wherein said silicone elastomer is poly(dimethoxy siloxane).

26. The method defined in claim 25, wherein the additives of said composition comprises a cross-linking agent, a diluent, and a radiopaque material.

27. The method defined in claim 26, wherein said composition is packaged as a kit of parts, comprising filling a first container with said silicone elastomer, and filling a second container with said cross-linking agent.

28. The method defined in claim 27, wherein said kit of parts comprises a mixing-dispensing device, comprising said container and a temporary seal between the containers, wherein one container is provided with a movable stirrer.

29. The method defined in claim 27 wherein said kit of parts comprises a mixing-dispensing device, comprising said containers and a mixing channel at the exits of the two-containers, wherein said channel is provided with a static mixing element.

30. The method defined in claim 26, comprising the steps of thoroughly intermixing:
A. between about 60% and 85% by weight based upon the weight of the entire composition of poly(dimethyl siloxane);
B. between about 2% and 5% by weight based upon the weight of the entire composition of the cross-linking agent;
C. between about 100% and 20% by weight based upon the weight of the entire composition of the diluent; and
D. between about 10% and 20% by weight based upon the weight of the entire composition of the radiopaque material,
thereby forming the desired injection composition.

31. The method in defined in claim 1, comprising the steps of injecting the curable filler material in two or more stages.

32. The method in defined in claim 31, in which the first stage is directed to fill the initials defects or voids in the trabecular bone of the femoral head or to reinforce or strengthen a weakened area of the femoral head.

33. The method in defined in claim 32, in which the durometer of the resulting cured material is selected to provide increased strength and stability to the trabecular bone in the femoral head.

34. The method in defined in claim 31, in which the second stage is directed to fill the area of the femoral head directly beneath the cartilage, supporting the articular surface, said second stage injection occurring after the first material has cured during the initial procedure, or may be performed during a subsequent procedure.

35. The method in defined in claim 34, in which the durometer of the resulting cured material injected in the second stage is selected to provide increased flexibility, resilience, or a greater capacity to distribute the forces seen by the articulating surface (“shock-absorbing” characteristic).

36. The method in defined in claim 35, in which the durometer of the resulting cured material injected in the second stage is different from the durometer injected in the first stage.

37. The method in defined in claim 34, in which the material injected directly beneath the cartilage as the first stage procedure, followed by injection of material into the trabecular bone.

38. The method in defined in claim 31, in which the curable filler composition is injected through a needle which passes through the resilient, cured material which has been implanted in the first stage.

39. The method in defined in claim 19, in which the injection of the curable filler composition is applied through a needle or cannula which positioned in the femoral head through the use of a drill guide

40. The method in defined in claim 39, in which the injection of the curable filler composition is applied in combination with a drill sleeve fitted to a common drill guide system preferred by the surgical team.
